(19) United States
(12) Patent Application Publication
(10) Pub. No.: US 2024/0099689 A1
(43) Pub. Date: Mar. 28, 2024

(54) ULTRASOUND ENDOSCOPY

(71) Applicant: AMBU A/S, Ballerup (DK)

(72) Inventors: Søren Elmin DIEDERICHSEN, Frederiksberg (DK); Finn SONNENBORG, Frederikssund (DK); Morten SØRENSEN, Ballerup (DK)

(73) Assignee: AMBU A/S, Ballerup (DK)

(21) Appl. No.: 18/372,862

(22) Filed: Sep. 26, 2023

(30) Foreign Application Priority Data
Sep. 27, 2022 (EP) 22198097.2

(51) Int. Cl. A61B 8/12; A61B 8/00

(52) U.S. Cl. CPC A61B 8/12; A61B 8/445; A61B 8/4477; A61B 8/4494; A61B 2562/166

(57) ABSTRACT

An endoscope including a handle, an insertion tube, a first flexible cable and an ultrasound transducer for receiving and transmitting ultrasound, the ultrasound transducer including an ultrasound transducer array attached to a distal end of the insertion tube, the handle being attached to a proximal end of the insertion tube, the first flexible cable is extending from the handle and being electrically connected to the ultrasound transducer array. The ultrasound transducer includes an electrical circuit for receiving and providing electrical signals to the ultrasound transducer array. The electrical circuit includes a flexible base section, a plurality of first flexible electrical conductors, and a contact section comprising a plurality of second electrical contacts. The flexible base section and the plurality of first flexible electrical conductors is a single flexible printed circuit and the first flexible electrical conductors extend from the flexible base section and into the first flexible cable.

### CROSS-REFERENCE TO RELATED APPLICATIONS

This application claims priority from and the benefit of European Patent Application No. EP22198097.2, filed Sep. 27, 2022; the disclosure of the aforementioned application is incorporated by reference herein in its entirety.

### TECHNICAL FIELD

The present disclosure relates to an endoscope, a printed flexible electrical circuit, and a system.

### BACKGROUND

Endoscopes are widely used in hospitals for visually examining body cavities and obtaining samples of tissue identified as potentially pathological. An endoscope typically comprises an image capturing device arranged at the distal end of the endoscope either looking forward or to the side.

The image capturing device is however restricted by the limited penetration depth of visible light. To examining deeper structures other imaging modalities must be used. One possibility is to provide the endoscope with an ultrasound transducer. By using emission of ultrasound waves deeper structures may be examined.

A modern ultrasound transducer comprises a plurality of ultrasound transducer element such as 32 or more ultrasound transducer elements. Each ultrasound transducer element must be provided with an electrical conductor for providing and receiving electrical signals. Performing the required electrical wiring is however a difficult and time-consuming task. This is one of the reasons why it is complex and expensive to manufacture a ultrasound transducer. Ultrasound endoscopes are therefore significantly more expensive than regular endoscopes. Availability of endoscopic ultrasound is therefore limited for some patient groups.

Furthermore, endoscopes are difficult to clean and there exist the risk of cross-contamination, i.e. that a pathological condition is transferred between patients as result of improper cleaning. The most secure solution has been the introduction of single-use endoscopes. A single use endoscope eliminates any risk of cross-contamination. The cost of an ultrasound transducer has however made it difficult to provide single use ultrasound endoscopes. This is problematic since the provision of an ultrasound probe at the distal end of the endoscope makes the endoscope even harder to clean. Thus, it remains a problem to provide an improved ultrasound transducer.

### BRIEF DESCRIPTION OF THE DISCLOSURE

The tasks and objectives of the present disclosure are to eliminate or at least to reduce the disadvantages of the related art. In particular, problems addressed by the present disclosure include how to reduce the cost of a single-use ultrasound endoscope and how to reduce the cost sufficiently to enable high volume production and deployment so that single-use endoscopes can be used regardless of the economic circumstances of the patients.

According to a first aspect, the present disclosure relates to an endoscope. In one embodiment, the endoscope comprises a printed flexible electrical circuit according to a second aspect described below. The endoscope further comprises a handle, an insertion tube having a proximal end and a distal end, a first flexible cable and an ultrasound transducer for receiving and transmitting ultrasound, the ultrasound transducer comprising an ultrasound transducer array attached to the distal end of the insertion tube, the handle being attached to the proximal end of the insertion tube, the first flexible cable is extending from the handle and being electrically connected to the ultrasound transducer array and is connectable to an ultrasound processing apparatus and configured to provide the ultrasound processing apparatus with electrical signals generated by the ultrasound transducer array and receive electrical signals generated by the ultrasound processing apparatus for the ultrasound transducer array, and wherein the ultrasound transducer array comprises a plurality of ultrasound transducer elements, and wherein the ultrasound transducer comprises an electrical circuit for receiving and providing electrical signals to the plurality of ultrasound transducer elements, and a support structure having an upper surface facing the plurality of ultrasound transducer elements and extending in a direction parallel to a first axis. The electrical circuit comprises a flexible base section comprising a plurality of first electrical contacts each first electrical contact being connected to an ultrasound transducer element for providing and receiving electrical signals to/from the ultrasound transducer element and extending along a central axis being parallel to the first axis, the flexible base section being arranged on the upper surface of the support structure, a plurality of first flexible electrical conductors each first flexible electrical conductor being electrically connected to a first electrical contact, a contact section comprising a plurality of second electrical contacts each second electrical contact being electrically connected to a first electrical conductor and further electrically connected or connectable to a second electrical conductor or a transducer port of an ultrasound processing apparatus. The flexible base section and the plurality of first flexible electrical conductors is a single flexible printed circuit. The first flexible electrical conductors extend from the flexible base section and into the handle.

Consequently, by using an electrical circuit provided with a flexible base section having a plurality of electrical contacts and where a plurality of first flexible electrical conductors extend into the handle, the plurality of ultrasound transducer elements may be provided with an electrical connection in a single process step. This makes it simple to manufacture.

The flexible printed circuit may be made using Photolithography. An adhesive may be provided between the lower side of the ultrasound transducer array and the flexible base section. Correspondingly, an adhesive may be provided between the flexible base section and the upper surface of the support structure. After attachment to the plurality of ultrasound transduce elements the flexibility of the flexible base section is limited by the flexibility of the ultrasound transducer array.

The ultrasound transducer elements may be made from a piezo electric block. The piezoelectric block may be made of any kind of piezoelectric material such as lead zirconate titanate (PZT), Quartz, Barium titanate, Poly(vinylidene fluoride) (PVDF), or a piezoelectric composite material.

The ultrasound transducer may be provided with one or more acoustic matching layers configured to provide an acoustic impedance gradient between the acoustic impedance of the tissue and the acoustic impedance of the ultrasound transducer elements. The acoustic matching layer may be made of epoxy, polyurethane, or polystyrene.

The ultrasound transducer may be configured to generate ultrasound with a centre frequency between 1 MHz and 15 MHz, or 2 MHz and 8 MHz.

The plurality of ultrasound transducer elements may be arranged in a common reference plane and configured to image a single image plane.

The plurality of ultrasound transducer elements may have been pre-poled and metalized before being attached to flexible base section. As an example, one or two layers of a nickel-chromium alloy may be applied to the plurality of ultrasound transducer elements. Alternatively/additionally 1 or two layers of Au may be applied. The layer may preferably be thin layers having a thickness of 10-20 nm. The layers may improve the piezoelectric properties of the piezoelectric block.

The support structure is preferably made of a material configured to absorb ultrasound energy. The material may be a composite material. As an example, the material may be an epoxy mixed with tungsten powder.

The upper surface of the support structure may be a convex upper surface, i.e. the ultrasound transducer array may be a curvilinear ultrasound transducer array. Alternatively, the upper surface of the support structure may be planar, i.e. the ultrasound transducer array may be a linear array.

The ultrasound transducer may further comprise an acoustic lens for focusing the ultrasound energy. The acoustic lens may be configured to focus the ultrasound energy in the image plane.

In some embodiments, the plurality of first flexible electrical conductors for a first part of their length extends inside the insertion tube, for a second part of their length extends inside the handle, and for a third part of their length extend inside the first flexible cable.

In some embodiments, the first flexible cable comprises an ultrasound connector insertable into a transducer port of an ultrasound processing apparatus for providing electrical signals generated by the ultrasound transducer array to the ultrasound processing apparatus and receiving electrical signals generated by the ultrasound processing apparatus, wherein the plurality of first flexible electrical conductors extend throughout the entire length of the first flexible cable and the ultrasound connector is the contact section. Consequently, by using a single flexible printed circuit for the entire electrical connection between the ultrasound array and the ultrasound connector it becomes simpler to manufacture the endoscope.

In some embodiments, the endoscope further comprises an image capturing device and wherein the first flexible cable is further configured to receive electrical signals from the image capturing device. Consequently, by using a single flexible cable to transfer signals from both the ultrasound transducer and the image capturing device it may become simpler for a user to operate the endoscope.

In some embodiments, the plurality of first flexible electrical conductors are folded at least once to extend their length. Consequently, the cost of manufacturing the flexible printed circuit may be reduced.

In some embodiments, the upper surface is a convex upper surface, the plurality of first flexible electrical conductors are bent around the support structure at a first curve section and at a second curve section, the first curve section being separate from the second curve section. Consequently, by providing the electrical circuit with a plurality of flexible electrical conductors bent around the support structure at a first curve section and second curve section, a single electrical circuit may be used to transport the electrical signal to and from the plurality of electrical contacts as the electric circuit may conform to the curvature of the convex upper surface.

The plurality of first flexible electrical conductors may be bent around the support structure at more curve sections than the first curve section and the second curve section. As an example, the plurality of first flexible electrical conductors may be bend around the support structure at least at three, four, six, or eight curve sections.

In some embodiments, the support structure has a first side surface arranged in a plane being perpendicular to the first axis, the first side surface being connected to the convex upper surface and wherein the first curve section is arranged along the connection between the convex upper surface and the first side surface.

In some embodiments, the second curve section is arranged along the connection between the convex upper surface and the first side surface and divided from the first curve section by a gap. Consequently, it may become possible to guide a significant number (or all) of the flexible electrical conductors away from the convex upper surface of the support structure in a simple and compact manner.

In some embodiments, the support structure comprises a concave lower surface opposite to the convex upper surface, the first side surface is connected to the concave lower surface, the gap extends along the entire first side of the support structure, and wherein the plurality of the first flexible electrical conductors are further bent around the support structure at a third curve section and at a fourth curve section, the third curve section being separate from the fourth curve section by the gap, and wherein the third curve section and the fourth curve section are arranged along the connection between the first side surface and the concave lower surface.

Consequently, the concave lower surface may be utilized to provide space for directing the plurality of first flexible electrical conductors away from the ultrasound transducer elements. This may reduce the amount of space required by the electric circuit.

According to a second aspect, the present disclosure relates to a printed flexible electrical circuit for receiving and providing electrical signals to an ultrasound transducer array comprising a plurality of ultrasound transducer elements, the printed flexible electrical circuit comprising: a flexible base section comprising a plurality of first electrical contacts each first electrical contact being connectable to an ultrasound transducer element for providing and receiving electrical signals to the ultrasound transducer element and extending along a central axis being parallel to a first axis; a plurality of first flexible electrical conductors each first flexible electrical conductor being electrically connected to a first electrical contact; and wherein the plurality of first flexible electrical conductors are connectable to an ultrasound connector whereby the first flexible electrical conductors may be responsible for the entire electrical coupling between the ultrasound connector and the ultrasound transducer array.

In some embodiments, the plurality of first flexible electrical conductors have a length of at least 40 cm.

According to a third aspect, the present disclosure relates to a system comprising an endoscope as disclosed in relation to the first aspect of the disclosure and an ultrasound processing apparatus, wherein the endoscope comprises an image capturing device and the first flexible cable is connectable to the ultrasound processing apparatus.

Ultrasound processing apparatuses are well-known in the art. Ultrasound processing apparatuses are configured to generate the electrical signals needed by the ultrasound transducer elements to generate the ultrasound waves. The electrical signal should have a centre frequency substantially matching the centre frequency of the ultrasound transducer elements. Furthermore, the signal should have a length and amplitude securing that a sufficient SNR is achieved. Furthermore, the ultrasound processing apparatus is configured to generate the ultrasound images by processing the signals generated by the ultrasound transducer elements. The ultrasound imaging may be done by using ultrasound beamforming techniques such as delay and sum beamforming in receive. In transmit, the ultrasound energy may be focused in a particular point by applying a suitable delay to the individual ultrasound transducer elements. Alternatively, a plane wave or a diverging ultrasound wave may be used in transmit.

The ultrasound processing apparatus may be adapted to cause a display to display a live representation of the electrical signals received from the ultrasound transducer. The ultrasound processing apparatus may be couplable to a display. The ultrasound processing apparatus may comprise a display for displaying a live representation of the electrical signals received from the ultrasound transducer and/or the image capturing device.

The ultrasound processing apparatus may be a portable ultrasound processing apparatus.

In some embodiments, the first flexible cable is further configured to receive electrical signals from the image capturing device and provide the received electrical signals to the ultrasound processing apparatus, and wherein the ultrasound processing apparatus comprise one or more processing units configured to process both the signals received from the ultrasound transducer and the signals received from the image capturing device. Consequently, by using a single flexible cable to transfer signals from both the ultrasound transducer and the image capturing device it may becomes simpler for user to operate the endoscope.

The ultrasound processing apparatus may comprise one or more processing units configured to process both the signals received from the ultrasound transducer and the signals received from the image capturing device.

Alternatively, the ultrasound processing apparatus may comprise a first set of one or more processing units configured to process the signals received from the ultrasound transducer and a second set of one or more processing units configured to process the signals received form the image capturing device. The first set and second set of one or more processing units may be provided in the same housing or in separate housings where the separate housings are communicatively coupled.

In some embodiments, the system further comprises an video processing apparatus and wherein the endoscope further comprises a second flexible cable configured to receive electrical signals from the image capturing device and provide the received electrical signals to the video processing apparatus.

Consequently, it may become simpler to connect the endoscope to a separate ultrasound processing apparatus and video processing apparatus.

The video processing apparatus may be adapted to cause a display to display a live representation of the electrical signals received from image capturing device. The video processing apparatus may be couplable to a display. The video processing apparatus may comprise a display for displaying a live representation of the electrical signals received from the image capturing device.

The video processing apparatus may be a portable video processing apparatus.

In some embodiments, the first flexible cable and the second flexible cable are connected along a first part of their length and disconnected along a second part of their length.

Consequently, it may both become simpler for the user to operate the endoscope and connect the cables to a separate ultrasound processing apparatus and video processing apparatus.

The first flexible cable may be attached to the second cable using an adhesive. Additionally/alternatively an outer cable may be arranged around the first flexible cable and the second flexible cable along the first part of their length.

The different aspects of the present disclosure can be implemented in different ways including as an endoscope, a printed flexible electrical circuit, and an endoscope system described above and in the following, each yielding one or more of the benefits and advantages described in connection with at least one of the aspects described above, and each having one or more preferred embodiments corresponding to the preferred embodiments described in connection with at least one of the aspects described above and/or disclosed in the dependent claims. Furthermore, it will be appreciated that embodiments described in connection with one of the aspects described herein may equally be applied to the other aspects.

### BRIEF DESCRIPTION OF THE DRAWINGS

The above and/or additional objects, features and advantages of the present disclosure, will be further elucidated by the following illustrative and non-limiting detailed description of embodiments of the present disclosure, with reference to the appended drawings, wherein:

FIGS. 1A and 1B show schematically an endoscope according to an embodiment of the disclosure.

FIGS. 2A and 2B show schematically a support structure for an ultrasound transducer according to an embodiment of the disclosure.

FIGS. 3A, 3B and 3C show schematically a support structure and an electrical circuit for an ultrasound transducer according to an embodiment of the disclosure.

FIGS. 4A, 4B and 4C show schematically a support structure and an electrical circuit for an ultrasound transducer according to an embodiment of the disclosure.

FIGS. 5A and 5B show schematically a support structure and an electrical circuit for an ultrasound transducer according to an embodiment of the disclosure.

FIG. 6 shows schematically an ultrasound transducer according to an embodiment of the disclosure.

FIG. 7 shows schematically a system according to an embodiment of the disclosure.

FIG. 8 shows schematically a system according to an embodiment of the disclosure.

### BRIEF DESCRIPTION OF THE DRAWINGS

In the following description, reference is made to the accompanying figures, which show by way of illustration how the embodiments of the present disclosure may be practiced.

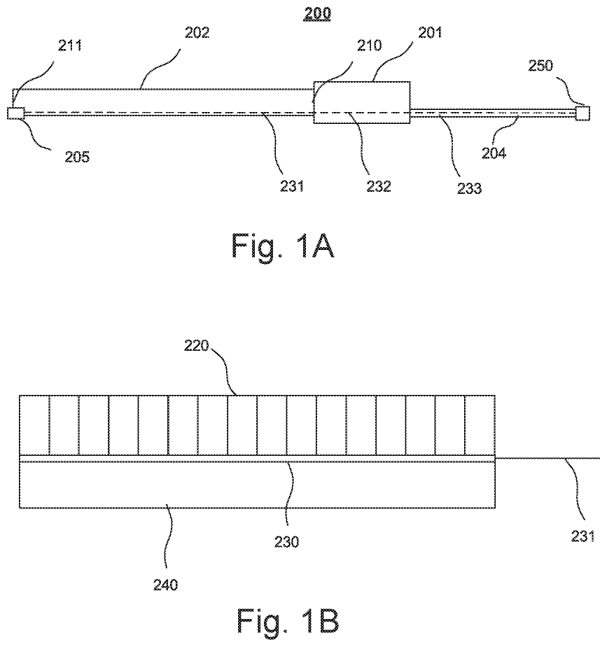

FIGS. 1A and 1B show schematically an endoscope provided with an ultrasound transducer according to an embodiment of the present disclosure. FIG. 1A shows a view of the endoscope, and FIG. 1B shows a close-up of a part of the ultrasound transducer. The endoscope (200) comprises a handle (201), an insertion tube (202) having a proximal end (210) and a distal end (211), a first flexible cable (204) and an ultrasound transducer (205) for receiving and transmitting ultrasound. The ultrasound transducer (205) comprising an ultrasound transducer array (220) attached to the distal end (211) of the insertion tube (202), the handle (201) being attached to the proximal end (210) of the insertion tube (202), the first flexible cable (204) is extending from the handle (201) and being electrically connected to the ultrasound transducer array (220) and is connectable to an ultrasound processing apparatus (not shown) and configured to provide the ultrasound processing apparatus with electrical signals generated by the ultrasound transducer array (220) and receive electrical signals generated by the ultrasound processing apparatus for the ultrasound transducer array.

The ultrasound transducer array (220) comprises a plurality of ultrasound transducer elements, an electrical circuit (230)-(233) for receiving and providing electrical signals to the plurality of ultrasound transducer elements, and a support structure (240) having an upper surface facing the plurality of ultrasound transducer elements and extending in a direction parallel to a first axis (not shown). The electrical circuit (230-233) comprises a flexible base section (230), a plurality of first flexible electrical conductors (231-233), and a contact section (250). The individual first flexible electrical conductors are not shown. The flexible base section (230) comprises a plurality of first electrical contacts (not shown) each first electrical contact being connected to an ultrasound transducer element for providing and receiving electrical signals to/from the ultrasound transducer element and extending along a central axis (not shown) being parallel to the first axis, the flexible base section (230) being arranged on the upper surface of the support structure (240).

Each of the plurality of first flexible electrical conductors (231-233) being electrically connected to a first electrical contact. The contact section (250) comprising a plurality of second electrical contacts each second electrical contact being electrically connected to a first electrical conductor (230-233) and further electrically connected or connectable to a second electrical conductor or a transducer port of an ultrasound processing apparatus. The flexible base section (230) and the plurality of first flexible electrical conductors (231-233) is a single flexible printed circuit and the first flexible electrical conductors (231-233) extend from the flexible base section (230) and into the handle (201). In this embodiment, the plurality of first flexible electrical conductors (231-233) for a first part of their length (231) extends inside the insertion tube (202), for a second part of their length (232) extends inside the handle (201), and for a third part of their length (233) extend inside the first flexible cable.

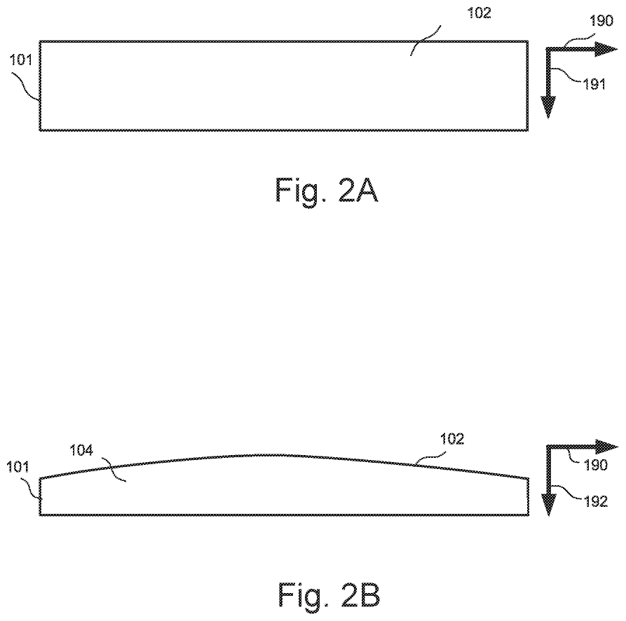

FIGS. 2A and 2B show schematically a support structure (101) for an ultrasound transducer according to an embodiment of the disclosure, where FIG. 2A shows a top view and FIG. 2B shows a side view. The support structure (101) has a convex upper surface (102) for facing a plurality of ultrasound transducer elements. The convex upper surface (102) extends in a direction parallel to a first axis (191). There is further shown a second axis (190) and a third axis (192). The second axis (190) being perpendicular to the first axis (191), and the third axis (192) being perpendicular to both the first axis (191) and the second axis (190). In addition to supporting the ultrasound transducer elements, the support structure may assist in absorbing ultrasound energy. Thus, the support structure (101) is preferably made of a material configured to absorb ultrasound energy. The material may be a composite material. The material may be an epoxy mixed with tungsten powder.

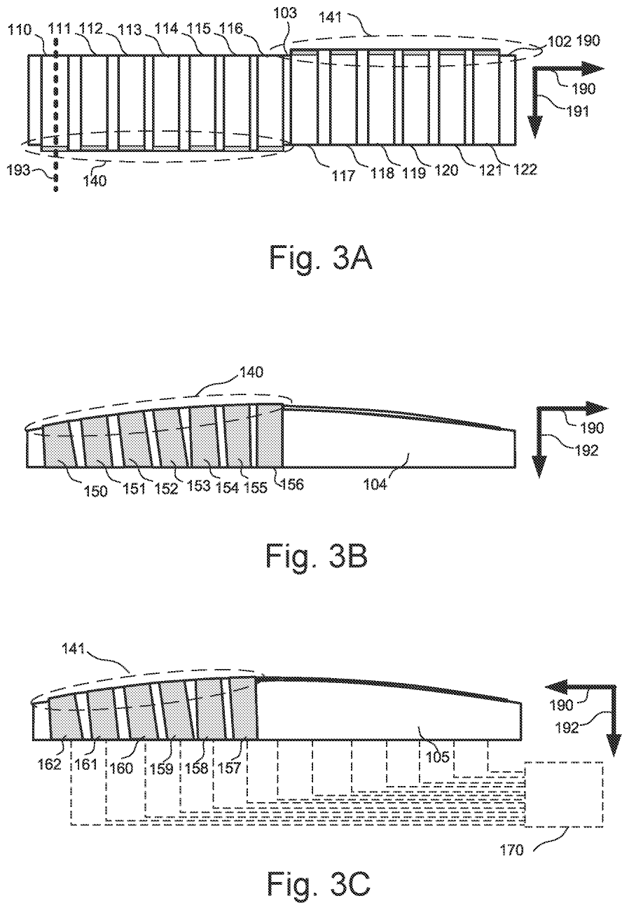

FIGS. 3A, 3B and 3C show schematically a support structure (101) and an electrical circuit for an ultrasound transducer according to an embodiment of the disclosure, where FIG. 3A shows a top view, FIG. 3B shows a first side view, and FIG. 3C shows a second side view. The support structure (101) corresponds to the support structure (101) shown in FIGS. 2A and 2B. The electrical circuit comprises a flexible base section (103) comprising a plurality of first electrical contacts (110-122) each first electrical contact (110-122) being connectable to an ultrasound transducer element for providing and receiving electrical signals to/from the ultrasound transducer element.

Each of the first electrical contacts (110-122) extends along a central axis (193) being parallel to the first axis (191). For simplicity only the central axis (193) for the first electrical contact 110 is shown. The flexible base section (103) is arranged on the convex upper surface (102) of the support structure (101). The first electrical contacts (110-122) should be made of a material having a high electrical conductivity such as Cu, Al, Ag, or Au. The area of the flexible base section (103) between the plurality of first electrical contacts (110-122) should be electrically isolating to prevent crosstalk between the ultrasound transducer elements. Correspondingly, the flexible base section (103) is preferably provided with one or more electrically isolating lower layers. For simplicity only 13 first electrical contacts (110-122) are shown. However, in other embodiments there may be at least 16, 32 or 64 first electrical contacts.

The electrical circuit further comprises a plurality of first flexible electrical conductors (150-162) each first flexible electrical conductor being electrically connected to a first electrical contact (110-122). The plurality of first flexible electrical conductors (150-162) are responsible for transferring electrical signals from the plurality of first electrical contacts (110-122) towards an ultrasound processing apparatus and transferring electrical signals from an ultrasound processing apparatus towards the plurality of first electrical contacts (110-122). The plurality of first flexible electrical conductors (150-162) should be made of a material having a high electrical conductivity such as Cu, Al, Ag, or Au. The area between the plurality of first flexible electrical conductors (150-162) should be electrically isolating to prevent cross-talk between the ultrasound transducer elements. Correspondingly, the plurality of first flexible electrical conductors (150-162) are preferably provided with one or more electrically isolating upper layers and one or more electrically isolating lower layers. The plurality of first flexible electrical conductors (150-162) are bent around the support structure (101) at a first curve section (140) and at a second curve section (141), the first curve section (140) being separate from the second curve section (141). The plurality of first flexible electrical conductors comprises a first group of flexible electrical conductors (150-156) and a second group of flexible electrical conductors (157-162). The support structure (101) has a first side surface (104) being arranged in a plane (not shown) being perpendicular to the first axis (191), the first side surface (104) being connected to the convex upper surface (102) and wherein the first curve section (140) is arranged along the connection between the convex upper surface (102) and the first side surface (104).

The support structure (101) has a second side surface (105) opposite to the first side surface (104), the second side surface (105) being arranged in a plane (not shown) being perpendicular to the first axis (191), the second side surface (105) being connected to the convex upper surface (102), the second curve section (141) is arranged along the connection between the convex upper surface (102) and the second side surface (105). The first group of flexible electrical conductors (150-156) are bent around the first curve section (140) and the second group of flexible electrical conductors (157-162) are bent around the second curve section (141). Only the first part of the plurality of first electrical conductors is shown in FIGS. 3A and 3B. In FIG. 3C are the remaining part of the first electrical conductors schematically illustrated by dashed lines. The electrical circuit further comprises a contact section (170) comprising a plurality of second electrical contacts each second electrical contact being electrically connected to a first electrical conductor (150-162) and further electrical connected or connectable to a second electrical conductor or a transducer port of an ultrasound processing apparatus.

In some embodiments, the contact section (170) is the ultrasound connector that is inserted into the transducer port of an ultrasound processing apparatus and the first electrical conductors (150-162) are responsible for the entire electrical coupling between the plurality of first electrical contacts (110-122) and the ultrasound connector.

In other embodiments, each of the plurality of second electrical contacts of the contact section (170) are connected to a second electrical conductor and the first electrical conductors (150-162) are only responsible for part of the electrical coupling between the plurality of first electrical contacts (110-122) and the ultrasound connector. The second electrical conductor may be a coaxial cable or a printed flexible electrical conductor. In one embodiment, the contact section is located in the handle of the endoscope and the second electrical conductor extends into the first flexible cable.

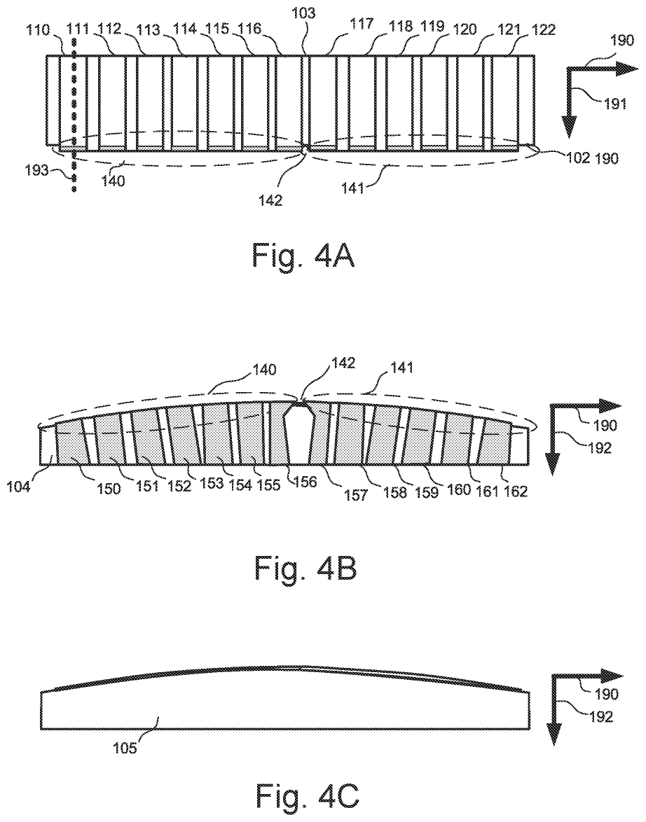

FIGS. 4A, 4B and 4C show schematically a support structure (101) and an electrical circuit for an ultrasound transducer according to an embodiment of the disclosure, where FIG. 4A shows a top view, FIG. 4B shows a first side view, and FIG. 4C shows a second side view. The support structure (101) corresponds to the support structure (101) shown in FIG. 2A and 2B. The electrical circuit comprises a flexible base section (103) comprising a plurality of first electrical contacts (110-122) each first electrical contact (110-122) being connectable to an ultrasound transducer element for providing and receiving electrical signals to/from the ultrasound transducer element. Each of the first electrical contacts (110-122) extends along a central axis (193) being parallel to the first axis (191). For simplicity only the central axis (193) for the first electrical contact 110 is shown. The flexible base section (103) is arranged on the convex upper surface (102) of the support structure (101). The electrical circuit further comprises a plurality of first flexible electrical conductors (150-162) each first flexible electrical conductor being electrically connected to a first electrical contact (110-122). The plurality of first flexible electrical conductors (150-162) are responsible for transferring electrical signals from the plurality of first electrical contacts (110-122) towards an ultrasound processing apparatus and transferring electrical signals from an ultrasound processing apparatus towards the plurality of first electrical contacts (110-122).

The support structure (101) has a first side surface (104) arranged in a plane (not shown) being perpendicular to the first axis (191), the first side surface (104) being connected to the convex upper surface (102). The plurality of first flexible electrical conductors (150-162) are bent around the support structure (101) at a first curve section (140) and at a second curve section (141), the first curve section (140) being separate from the second curve section (141). The first curve section (140) and the second curve section (141) are arranged along the connection between the convex upper surface (102) and the first side surface (104) and divided by a gap (142). Only the first part of the plurality of first electrical conductors is shown in FIGS. 4A, 4B and 4C. The electrical circuit may further comprise a contact section (170) comprising a plurality of second electrical contacts each second electrical contact being electrically connected to a first electrical conductor (150-162) and further electrical connected or connectable to a second electrical conductor or a transducer port of an ultrasound system.

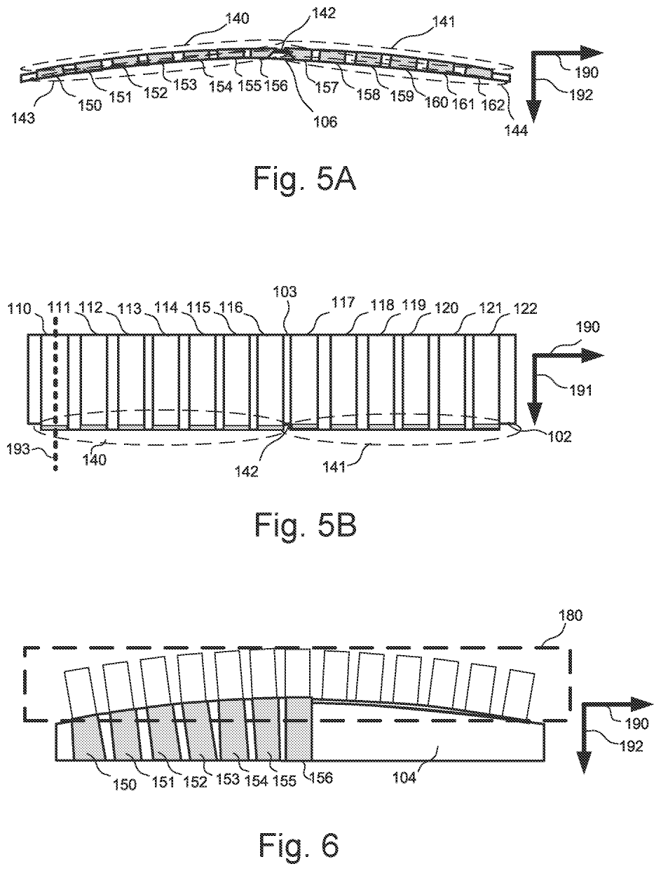

FIGS. 5A and 5B show schematically a support structure (101) and an electrical circuit for an ultrasound transducer according to an embodiment of the disclosure, where FIG. 5A shows a top view and FIG. 5B shows a side view. The embodiment shown in FIGS. 5A and 5B corresponds to the embodiment shown in FIGS. 4A, 4B and 4C, with the difference that the support structure (101) comprises a concave lower surface (106) opposite to the convex upper surface (102), the first side surface (104) is connected to the concave lower surface (106) and the gap (142) extends along the entire first side (104) of the support structure (101). The plurality of first flexible electrical conductors (150-162) are further bent around the support structure at a third curve section (143) and at a fourth curve section (144), the third curve section (143) being separate from the fourth curve section (144) by the gap (142), and wherein the third curve section (143) and the fourth curve section (144) are arranged along the connection between the first side surface (104) and the concave lower surface (106). Consequently, the concave lower surface may be utilized to provide space for directing the plurality of first flexible electrical conductors away from the ultrasound transducer elements. This may reduce the amount of space required by the electric circuit.

FIG. 6 shows schematically an ultrasound transducer according to an embodiment of the disclosure. The ultrasound transducer comprises a support structure and an electrical circuit corresponding to the support structure and the electrical circuit disclosed in relation to FIG. 3A, 3B and 3C. Furthermore, the ultrasound transducer comprises a convex ultrasound transducer array comprising a plurality of ultrasound transducer elements (180), where each first electrical contact (110-122) is connected to an ultrasound transducer element for providing and receiving electrical signals to/from the ultrasound transducer element.

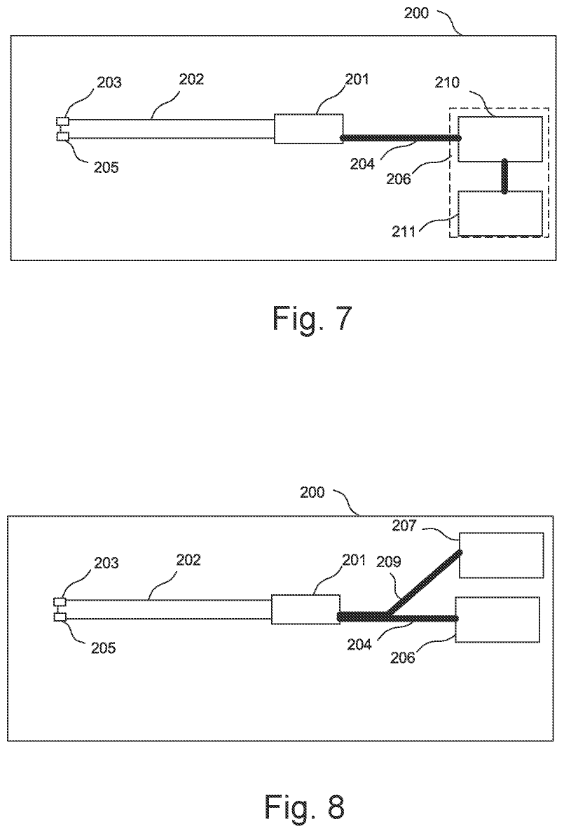

FIG. 7 shows schematically a system (200) comprising an endoscope and an ultrasound processing apparatus (206) according to an embodiment of the disclosure. The endoscope comprises a handle (201), an insertion tube (202), an image capturing device (203), a first flexible cable (204) and an ultrasound transducer (205). The ultrasound transducer may be an ultrasound transducer as disclosed in FIGS. 1-6. The handle (201) is attached to a proximal end of the insertion tube (202) and the image capturing device (203) and the ultrasound transducer (205) are attached to a distal end of the insertion tube (202). The first flexible cable (204) is extending from the handle (201) and is electrically connected to the ultrasound transducer (205) and wherein the first flexible cable is connected to the ultrasound processing apparatus (206) and configured to provide the ultrasound processing apparatus (206) with electrical signals generated by the ultrasound transducer (205) and receive electrical signals generated by the ultrasound processing apparatus (206) for the ultrasound transducer (205). In this embodiment, the first flexible cable (204) is further configured to receive electrical signals from the image capturing device (203) and provide the received electrical signals to the ultrasound processing apparatus (206). The ultrasound processing apparatus (206) comprises a first processing unit (210) configured to process electrical signals generated by the ultrasound transducer (205) and a second processing unit (211) configured to process image signals recorded by the image capturing device (203).

FIG. 8 shows schematically a system (200) comprising an endoscope an ultrasound processing apparatus (206), and an imaging unit (207) according to an embodiment of the disclosure. The endoscope comprises a handle (201), an insertion tube (202), an image capturing device (203), a first flexible cable (204), a second flexible cable (209) and an ultrasound transducer (205). The ultrasound transducer (205) may be an ultrasound transducer as disclosed in FIGS. 1-6. The handle (201) is attached to a proximal end of the insertion tube (202) and the image capturing device (203) and the ultrasound transducer (205) are attached to a distal end of the insertion tube (202). The first flexible cable (204) is extending from the handle (201) and is electrically connected to a plurality of ultrasound transducer elements via an electrical circuit and wherein the first flexible cable is connected to the ultrasound processing apparatus (206) and configured to provide the ultrasound processing apparatus (206) with electrical signals generated by the ultrasound transducer (205) and receive electrical signals generated by the ultrasound processing apparatus (206) for the ultrasound transducer (205). In this embodiment, the second flexible cable (209) is configured to receive electrical signals from the image capturing device (203) and provide the received electrical signals to an imaging unit, or video processing apparatus, (209). Example video processing apparatus are described in commonly owned U.S. Pat. Nos. 11,109,741 and 11,583,164, both incorporated by reference herein in their entirety.

Embodiments of the present disclosure, variations thereof, and examples thereof are set out in the following items:
1. An endoscope comprising a handle, an insertion tube having a proximal end and a distal end, a first flexible cable and an ultrasound transducer for receiving and transmitting ultrasound, the ultrasound transducer comprising an ultrasound transducer array attached to the distal end of the insertion tube, the handle being attached to the proximal end of the insertion tube, the first flexible cable is extending from the handle and being electrically connected to the ultrasound transducer array and is connectable to an ultrasound processing apparatus and configured to provide the ultrasound processing apparatus with electrical signals generated by the ultrasound transducer array and receive electrical signals generated by the ultrasound processing apparatus for the ultrasound transducer array, and wherein the ultrasound transducer array comprises a plurality of ultrasound transducer elements (180), and wherein the ultrasound transducer comprises an electrical circuit for receiving and providing electrical signals to the plurality of ultrasound transducer elements (180), and a support structure (101) having an upper surface (102) facing the plurality of ultrasound transducer elements (180) and extending in a direction parallel to a first axis (191), wherein the electrical circuit comprises: a flexible base section (103) comprising a plurality of first electrical contacts (110-122) each first electrical contact (110-122) being connected to an ultrasound transducer element for providing and receiving electrical signals to/from the ultrasound transducer element and extending along a central axis (193) being parallel to the first axis (191), the flexible base section (103) being arranged on the upper surface (102) of the support structure (101); a plurality of first flexible electrical conductors (150-162) each first flexible electrical conductor being electrically connected to a first electrical contact (110-122); and a contact section comprising a plurality of second electrical contacts each second electrical contact being electrically connected to a first electrical conductor (150-162) and further electrically connected or connectable to a second electrical conductor or a transducer port of an ultrasound processing apparatus, wherein the flexible base section and the plurality of first flexible electrical conductors is a single flexible printed circuit and wherein the first flexible electrical conductors (150-162) extend from the flexible base section (103) and into the handle.
2. An endoscope according to item 1, wherein the plurality of first flexible electrical conductors (150-162) for a first part of their length extends inside the insertion tube, for a second part of their length extends inside the handle, and for a third part of their length extend inside the first flexible cable.
3. An endoscope according to item 2, wherein the first flexible cable comprises an ultrasound connector insertable into a transducer port of an ultrasound processing apparatus for providing electrical signals generated by the ultrasound transducer array to the ultrasound processing apparatus and receiving electrical signals generated by the ultrasound processing apparatus, wherein the plurality of first flexible electrical conductors (150-162) extend throughout the entire length of the first flexible cable and the ultrasound connector is the contact section.
4. An endoscope according to item 3, wherein the endoscope further comprises an image capturing device and wherein the first flexible cable is further configured to receive electrical signals from the image capturing device.
5. An endoscope according to any one of items 1 to 4, wherein the plurality of first flexible electrical conductors (150-162) are folded at least once to extend their length.
6. An endoscope according to any one of items 1 to 5, wherein the upper surface (102) is a convex upper surface, the plurality of first flexible electrical conductors (150-162) are bent around the support structure (101) at a first curve section (140) and at a second curve section (141), the first curve section (140) being separate from the second curve section (141).
7. An endoscope according to item 6, wherein the support structure (101) has a first side surface (104) arranged in a plane being perpendicular to the first axis (191), the first side surface (104) being connected to the convex upper surface (102) and wherein the first curve section (140) is arranged along the connection between the convex upper surface (102) and the first side surface (104).
8. An endoscope according to item 7, wherein the second curve section (141) is arranged along the connection between the convex upper surface and the first side surface (104) and divided from the first curve section (140) by a gap (142).
9. An endoscope according to item 8, wherein the support structure comprises a concave lower surface (106) opposite to the convex upper surface (102), the first side surface (104) is connected to the concave lower surface (106), the gap (142) extends along the entire first side (104) of the support structure (101), and wherein the plurality of the first flexible electrical conductors (150-162) are further bent around the support structure at a third curve section (143) and at a fourth curve section (144), the third curve section (143) being separate from the fourth curve section (144) by the gap (142), and wherein the third curve section (143) and the fourth curve section (144) are arranged along the connection between the first side surface (104) and the concave lower surface (106).
10. A printed flexible electrical circuit for receiving and providing electrical signals to an ultrasound transducer array comprising a plurality of ultrasound transducer elements, the printed flexible electrical circuit comprising: a flexible base section (103) comprising a plurality of first electrical contacts (110-122) each first electrical contact (110-122) being connectable to an ultrasound transducer element for providing and receiving electrical signals to the ultrasound transducer element and extending along a central axis (193) being parallel to a first axis (191); and a plurality of first flexible electrical conductors (150-162) each first flexible electrical conductor being electrically connected to a first electrical contact (110-122), wherein the plurality of first flexible electrical conductors (150-162) are connectable to an ultrasound connector whereby the first flexible electrical conductors may be responsible for the entire electrical coupling between the ultrasound connector and the ultrasound transducer array.
11. A printed flexible electrical circuit wherein the plurality of first flexible electrical conductors (150-162) have a length of at least 40 cm.
12. A system comprising an endoscope according to any one of items 1 to 9 and an ultrasound processing apparatus, wherein the endoscope comprises an image capturing device and the first flexible cable is connectable to the ultrasound processing apparatus.
13. A system according to item 12, wherein the first flexible cable is further configured to receive electrical signals from the image capturing device and provide the received electrical signals to the ultrasound processing apparatus, and wherein the ultrasound processing apparatus comprise one or more processing units configured to process both the signals received from the ultrasound transducer and the signals received from the image capturing device.
14. A system according to item 13, wherein the system further comprises an video processing apparatus and wherein the endoscope further comprises a second flexible cable configured to receive electrical signals from the image capturing device and provide the received electrical signals to the video processing apparatus.

Although some embodiments have been described and shown in detail, the invention is not restricted to them, but may also be embodied in other ways within the scope of the subject matter defined in the following claims. In particular, it is to be understood that other embodiments may be utilised and structural and functional modifications may be made without departing from the scope of the present invention.

In device claims enumerating several means, several of these means can be embodied by one and the same item of hardware. The mere fact that certain measures are recited in mutually different dependent claims or described in different embodiments does not indicate that a combination of these measures cannot be used to advantage.

It should be emphasized that the term “comprises/comprising” when used in this specification is taken to specify the presence of stated features, integers, steps or components but does not preclude the presence or addition of one or more other features, integers, steps, components or groups thereof.

We claim:

1. An endoscope comprising:
an insertion tube having a proximal end and a distal end;
a handle attached to the proximal end of the insertion tube;
an ultrasound transducer attached to the distal end of the insertion tube and comprising an electrical circuit, a support structure, and an ultrasound transducer array; and
a first flexible cable electrically connected to the ultrasound transducer array and connectable to an ultrasound processing apparatus, the first flexible cable being configured to provide the ultrasound processing apparatus with electrical signals generated by the ultrasound transducer array and to provide electrical signals generated by the ultrasound processing apparatus to the ultrasound transducer array,
wherein the ultrasound transducer array comprises a plurality of ultrasound transducer elements,
wherein the support structure comprises an upper surface facing the plurality of ultrasound transducer elements and extending in a direction parallel to a first axis,
wherein the electrical circuit is configured to receive from and provide electrical signals to the plurality of ultrasound transducer elements and comprises:
a flexible base section comprising a plurality of first electrical contacts, each first electrical contact being connected to an ultrasound transducer element of the plurality of ultrasound transducer elements and extending along a central axis that is parallel to the first axis, the flexible base section being arranged on the upper surface of the support structure,
a plurality of first flexible electrical conductors, each first flexible electrical conductor being electrically connected to a first electrical contact, and
a contact section comprising a plurality of second electrical contacts, each second electrical contact being electrically connected to a first electrical conductor and further electrically connected or connectable to a second electrical conductor or a transducer port of an ultrasound processing apparatus,
wherein the flexible base section and the plurality of first flexible electrical conductors is a single flexible printed circuit, and
wherein the first flexible electrical conductors extend from the flexible base section and into the handle.

2. The endoscope of claim 1, wherein the plurality of first flexible electrical conductors for a first part of their length extend inside the insertion tube, for a second part of their length extend inside the handle, and for a third part of their length extend inside the first flexible cable.

3. The endoscope of claim 2, wherein the first flexible cable comprises an ultrasound connector insertable into a transducer port of an ultrasound processing apparatus for providing electrical signals generated by the ultrasound transducer array to the ultrasound processing apparatus and receiving electrical signals generated by the ultrasound processing apparatus, and wherein the plurality of first flexible electrical conductors extend throughout the entire length of the first flexible cable and the ultrasound connector is the contact section.

4. The endoscope of claim 3, wherein the endoscope further comprises an image capturing device, and wherein the first flexible cable is further configured to receive electrical signals from the image capturing device.

5. The endoscope of claim 4, wherein the plurality of first flexible electrical conductors are folded at least once to extend their length.

6. The endoscope of claim 1, wherein the upper surface is a convex upper surface, the plurality of first flexible electrical conductors are bent around the support structure at a first curve section and at a second curve section, the first curve section being separate from the second curve section.

7. The endoscope of claim 6, wherein the support structure has a first side surface arranged in a plane being perpendicular to the first axis, the first side surface being connected to the convex upper surface, and wherein the first curve section is arranged along the connection between the convex upper surface and the first side surface.

8. The endoscope of claim 7, wherein the second curve section is arranged along the connection between the convex upper surface and the first side surface and divided from the first curve section by a gap.

9. The endoscope of claim 8, wherein the support structure comprises a concave lower surface opposite to the convex upper surface, the first side surface is connected to the concave lower surface, the gap extends along the entire first side of the support structure, and wherein the plurality of the first flexible electrical conductors are further bent around the support structure at a third curve section and at a fourth curve section, the third curve section being separate from the fourth curve section by the gap, and wherein the third curve section and the fourth curve section are arranged along the connection between the first side surface and the concave lower surface.

10. The endoscope of claim 1, wherein the plurality of first flexible electrical conductors are folded at least once to extend their length.

11. The endoscope of claim 1, further comprising an image sensor, wherein the plurality of first flexible electrical conductors for a first part of their length extend inside the insertion tube, for a second part of their length extend inside the handle, and for a third part of their length extend inside the first flexible cable, wherein the first flexible cable is further configured to receive electrical signals from the image sensor, wherein the upper surface is a convex upper surface, the plurality of first flexible electrical conductors are bent around the support structure at a first curve section and at a second curve section, the first curve section being separate from the second curve section, wherein the support structure has a first side surface arranged in a plane being perpendicular to the first axis, the first side surface being connected to the convex upper surface, and wherein the first curve section is arranged along the connection between the convex upper surface and the first side surface, wherein the second curve section is arranged along the connection between the convex upper surface and the first side surface and divided from the first curve section by a gap, wherein the support structure comprises a concave lower surface opposite to the convex upper surface, the first side surface is connected to the concave lower surface, the gap extends along the entire first side of the support structure, and wherein the plurality of the first flexible electrical conductors are further bent around the support structure at a third curve section and at a fourth curve section, the third curve section being separate from the fourth curve section by the gap, and wherein the third curve section and the fourth curve section are arranged along the connection between the first side surface and the concave lower surface, and wherein the plurality of first flexible electrical conductors are folded at least once to extend their length.

12. The endoscope of claim 1, further comprising an image sensor, wherein the plurality of first flexible electrical conductors are folded at least once to extend their length, wherein the plurality of first flexible electrical conductors for a first part of their length extend inside the insertion tube, for a second part of their length extend inside the handle, and for a third part of their length extend inside the first flexible cable, and wherein the first flexible cable is further configured to receive electrical signals from the image sensor.

13. A system comprising:
an endoscope according to claim 1; and
an ultrasound processing apparatus,
wherein the endoscope comprises an image capturing device and the first flexible cable is connectable to the ultrasound processing apparatus.

14. The system of claim 13, wherein the first flexible cable is further configured to receive electrical signals from the image capturing device and provide the received electrical signals to the ultrasound processing apparatus, and wherein the ultrasound processing apparatus comprises one or more processing units configured to process both the signals received from the ultrasound transducer and the signals received from the image capturing device.

15. The system of claim 13, wherein the system further comprises an video processing apparatus and wherein the endoscope further comprises a second flexible cable configured to receive electrical signals from the image capturing device and provide the received electrical signals to the video processing apparatus.

16. The system of claim 13, wherein the plurality of first flexible electrical conductors for a first part of their length extend inside the insertion tube, for a second part of their length extend inside the handle, and for a third part of their length extend inside the first flexible cable, wherein the first flexible cable is further configured to receive electrical signals from the image capturing device, wherein the upper surface is a convex upper surface, the plurality of first flexible electrical conductors are bent around the support structure at a first curve section and at a second curve section, the first curve section being separate from the second curve section, wherein the support structure has a first side surface arranged in a plane being perpendicular to the first axis, the first side surface being connected to the convex upper surface, and wherein the first curve section is arranged along the connection between the convex upper surface and the first side surface, wherein the second curve section is arranged along the connection between the convex upper surface and the first side surface and divided from the first curve section by a gap, wherein the support structure comprises a concave lower surface opposite to the convex upper surface, the first side surface is connected to the concave lower surface, the gap extends along the entire first side of the support structure, and wherein the plurality of the first flexible electrical conductors are further bent around the support structure at a third curve section and at a fourth curve section, the third curve section being separate from the fourth curve section by the gap, and wherein the third curve section and the fourth curve section are arranged along the connection between the first side surface and the concave lower surface, and wherein the plurality of first flexible electrical conductors are folded at least once to extend their length.

17. A printed flexible electrical circuit for receiving and providing electrical signals to an ultrasound transducer array comprising a plurality of ultrasound transducer elements, the printed flexible electrical circuit comprising:
a flexible base section comprising a plurality of first electrical contacts each first electrical contact being connectable to an ultrasound transducer element for providing and receiving electrical signals to the ultrasound transducer element and extending along a central axis being parallel to a first axis, and
a plurality of first flexible electrical conductors each first flexible electrical conductor being electrically connected to a first electrical contact,
wherein the plurality of first flexible electrical conductors are connectable to an ultrasound connector, the first flexible electrical conductors providing an entire electrical coupling between the ultrasound connector and the ultrasound transducer array.

18. The printed flexible electrical circuit of claim 14, wherein the plurality of first flexible electrical conductors have a length of at least 40 cm.
